(19) United States
(12) Patent Application Publication
(10) Pub. No.: US 2024/0215804 A1
(43) Pub. Date: Jul. 4, 2024

(54) ENDOSCOPE SUCTION VALVE

(71) Applicant: Gastrologic, LLC, San Diego, CA (US)

(72) Inventor: Michael Docherty, San Diego, CA (US)

(21) Appl. No.: 18/403,530

(22) Filed: Jan. 3, 2024

Related U.S. Application Data
(60) Provisional application No. 63/436,823, filed on Jan. 3, 2023.

(51) Int. Cl. A61B 1/00; A61B 1/015

(52) U.S. Cl. CPC A61B 1/00068; A61B 1/015

(57) ABSTRACT

The present disclosure provides for an endoscope apparatus. The apparatus may include a body forming a passage wall that defines an interior passage extending between a suction inlet and a suction outlet. The apparatus may further include a vent inlet in fluid communication with the interior passage, and a valve. The valve may be movable between a first position and a second position. When the plug is in the first position, the plug may close the vent inlet without occluding the interior passage, and when the plug is in the second position, the vent inlet may be open.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application is a non-provisional of and claims priority to U.S. Provisional Patent Application No. 63/436,823, filed Jan. 3, 2023, entitled ENDOSCOPE SUCTION VALVE, which is hereby incorporated by reference in its entirety.

### STATEMENT REGARDING FEDERALLY SPONSORED RESEARCH OR DEVELOPMENT

Not Applicable.

### REFERENCE TO SEQUENCE LISTING OR COMPUTER PROGRAM LISTING APPENDIX

Not Applicable.

### BACKGROUND

The present disclosure relates generally to endoscope suction apparatuses, and more particularly to a valve for controlling endoscopic suction.

Endoscopes are often used to allow a medical care provider to observe and/or treat cavities or hollow organs within a patient. A tip of an endoscopic device may be inserted directly into the organ or cavity in order to perform such practices. Depending on the procedure, air, water, or some other fluid within the organ or cavity may need to be removed using the endoscope. For example, water or some other fluid may be delivered through the tip of the endoscopic device in order to clean the organ or cavity, or to clean the tip (and various surgical tools implemented thereon). As another example, air may be provided through the tip for insufflation of the organ or cavity. Such provisions of water, air, or other fluid may need to be removed from the organ or cavity in order to proceed with the endoscopic operation. Moreover, even before the insertion of the endoscope, various fluids may already be existing within the organ or cavity that need to be removed as part of the operation itself. As such, systems and methods of suction are of paramount importance for many typical endoscopic procedure.

Conventional systems for endoscopic suction involve an endoscope with a valve that the medical care provider may operate in order to activate suction through the tip of the endoscope. In some systems, the valve may also be used to control a particular degree of suction (e.g., a volumetric rate of flow being drawn through the tip of the endoscope). Such systems typically involve an elongate stem that is operated similar to (or the same as) a needle valve, in that an elongate member (e.g., a stem) is moved relative to a suction channel (e.g., an interior passage of the endoscope) through which the fluid flows from a suction inlet to a suction outlet in response to a pressure drop constantly imparted by a suction device that interfaces with the suction outlet. Due to the constant activity of the suction device, conventional systems use the stem to block the channel, thereby cutting the inlet off from the suction generated at the suction outlet, while also opening a vent or some other aperture through which room air may instead be drawn to the suction outlet by the suction device.

There are numerous issues involved with present valves for controlling endoscopic suction. As an example, many present systems use a stem with an intermediate opening and internal duct through which the fluid being drawn from the suction inlet flows. Such configurations necessarily draw fluid through a passage that is smaller than the internal passage of the endoscope device itself, thus limiting the functional rate of suction and impairing the efficiency of the endoscopic suction. Moreover, such internal ducts within the stems are subject to clogging, further limiting the functional rate of suction, while also requiring periodic maintenance to remove the clogging. In some cases, even when a stem with an internal duct is not used, such systems still typically use a plug that occludes the internal passage of the endoscope, which causes similar issues related to clogging and the efficiency of the endoscopic suction.

What is needed then is an improved endoscope valve that addresses these and other problems associated with typical systems.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is an endoscope device. The endoscope device includes a body forming a passage wall that defines an interior passage extending between a suction inlet and a suction outlet. The endoscope device further includes a suction pump disposed on the body and configured to draw a flow of fluid from the interior passage to the suction outlet, as well as a vent inlet disposed on the body and extending through the passage wall to the interior passage at a location that is between the suction inlet and the suction outlet. Finally, the endoscope device further includes a valve that includes a plug that is movable between various positions relative to the interior passage. For example, the plug may be placed in a first position that closes the vent inlet. As another example, the plug may be placed in a second position where the vent is opened. In some embodiments, each of the first and second positions are outside of the interior passage. For example, the plug may be placed in these positions, such that the plug does not occlude the interior passage.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a cross-sectional view showing a configuration of an endoscope suction apparatus according to some embodiments of the present disclosure.

FIG. 2A is a cross-sectional view showing a configuration concerning suction of a conventional endoscope in an actuated state.

FIG. 2B is a cross-sectional view showing a configuration concerning suction of a conventional endoscope in a de-actuated state.

FIG. 3A is a perspective view showing an embodiment of a conventional endoscope suction valve with the stem in a retracted position.

FIG. 3B is a perspective view showing an embodiment of a conventional endoscope suction valve.

FIG. 3C is a perspective view showing an embodiment of a conventional endoscope suction valve with the stem in an extended position.

FIG. 4A is a cross-sectional view showing a configuration concerning suction of a conventional endoscope in a de-actuated state.

FIG. 4B is a cross-sectional view showing a configuration concerning suction of a conventional endoscope in an actuated state.

FIG. 5 is a cross-sectional view showing a configuration of an endoscope suction valve in a fully actuated state, according to some embodiments of the present disclosure.

FIG. 6 is a cross-sectional view showing a configuration of an endoscope suction valve in a fully actuated state, to according to some embodiments of the present disclosure.

FIG. 7 is a cross-sectional view showing a configuration of an endoscope suction valve in a partially actuated state, according to some embodiments of the present disclosure.

FIG. 8 is a cross-sectional view showing a configuration of an endoscope suction valve in a partially actuated state, according to some embodiments of the present disclosure.

FIG. 9 is a cross sectional view showing a configuration of an endoscope suction valve in a fully de-actuated state, according to some embodiments of the present disclosure.

FIG. 10 is a side elevated view of various configurations of a plug for an endoscope suction valve, according to some embodiments of the present disclosure.

FIG. 11 is a cross-sectional view showing a configuration of an endoscope suction valve in a fully actuated state with an elongated plug, according to some embodiments of the present disclosure.

FIG. 12 is a cross-sectional view showing a configuration of an endoscope suction valve in a fully actuated state with a coating on the plug, to according to some embodiments of the present disclosure.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

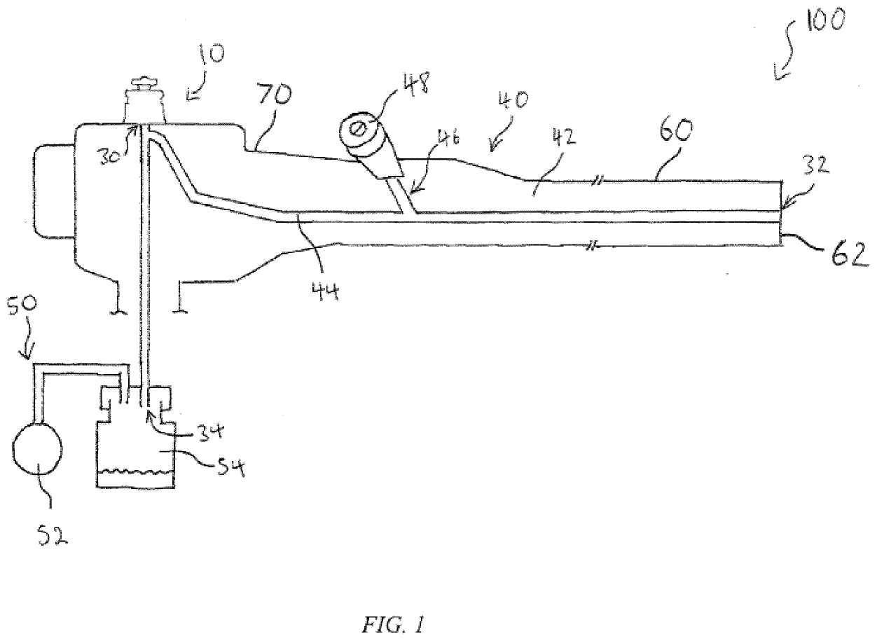

Referring now to FIG. 1, an endoscope suction apparatus (apparatus) 100 is shown, according to some embodiments of the present disclosure. As described in greater detail below, the apparatus 100 may be operated to perform endoscopic suction. The apparatus 100 includes a body 40 that forms a passage wall 42 that defines an interior passage 44 extending between a suction inlet 32 and a suction outlet 34. For example, the body 40 may include a distal insertion portion 60 that is inserted into a patient, terminating at a distal tip 62 that forms the suction inlet 32. During an exemplary procedure involving endoscopic suction, some or all of the insertion portion 60 may be inserted within a patient in order to draw a flow (of fluid, gas, solid matter, or some combination thereof) from the patient into the interior passage 44 via the suction inlet 32.

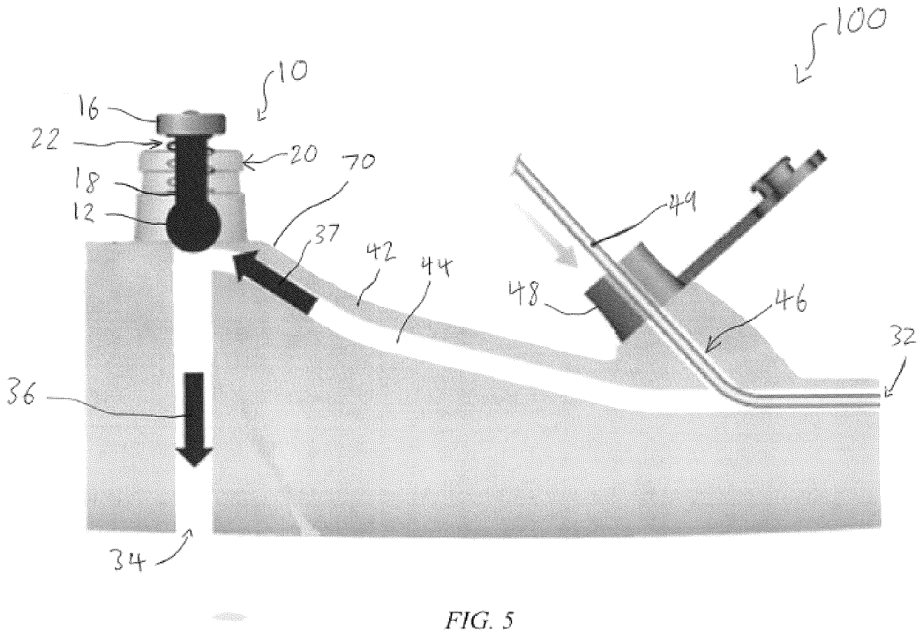

The body 40 may further include a proximal operation portion 70 interfacing with the distal insertion portion 60. A valve 10 and an instrument hub 48 may be mounted on the body 40 at the operation portion 70. As shown with reference to FIG. 5, the instrument hub 48 may be used to guide various endoscope instruments 49 (e.g., image sensing tools, forceps, etc.) through an instrument opening 46, into the interior passage 44, and through the suction inlet 32. In some embodiments, a vent inlet 30 may be disposed on the body 42. The vent inlet 30 may be in fluid communication with the interior passage 44. For example, the vent inlet 30 may extend through the passage wall 42 to the interior passage 44 at a location (along the path of the interior passage 44) that is between the suction inlet 32 and the suction outlet 34 (the proximal operation portion 70, for example). While the suction inlet 32 may be configured to draw a fluid, gaseous, or solid flow from the patient during the aforementioned exemplary procedure, the vent inlet 30 may be configured to draw air from the outside environment (e.g., “room air”).

In some embodiments, the apparatus 100 further includes a suction device 50, which engages the interior passage 44 at the suction outlet 34. The suction device 50 may include a pump 52 and a fluid storage tank 54. The pump 52 is configured to drive endoscopic suction by drawing a flow from the interior passage 44 to the suction outlet 34, at which point the flow may be stored in the fluid storage tank 54. During the exemplary procedure involving endoscopic suction, the pump 52 may be constantly operated to draw the flow from the interior passage 44 to the suction outlet 34. Of course, the interior passage 44 has at least two inlets (the suction inlet 32 and the vent inlet 30) through which the flow may be drawn. As described in greater detail below, the valve 10 may be used to control the endoscopic suction by determining from which inlet (the suction inlet 32 or the vent inlet 30) the flow is drawn or, in the case where the flow is drawn from each inlet, how much of the flow is drawn from each inlet. In practice, such control allows for dictating an amount of suction that is drawn from the patient (e.g., through the suction inlet 32 as opposed to the vent inlet 30).

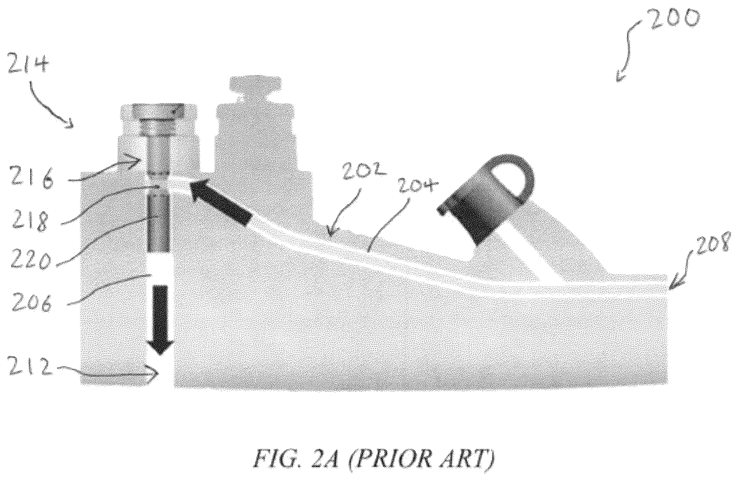

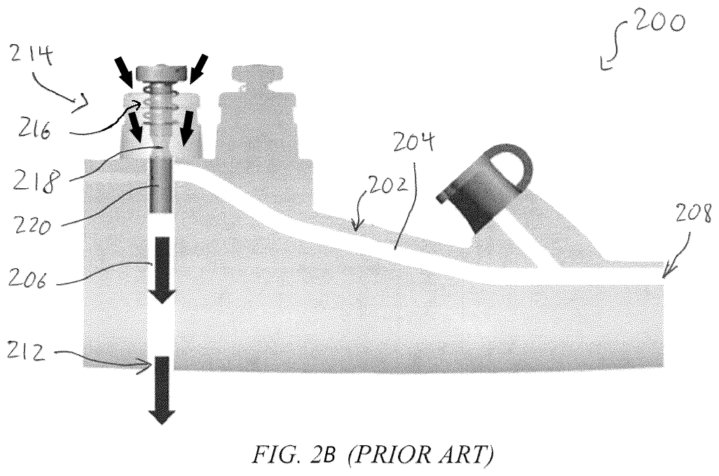

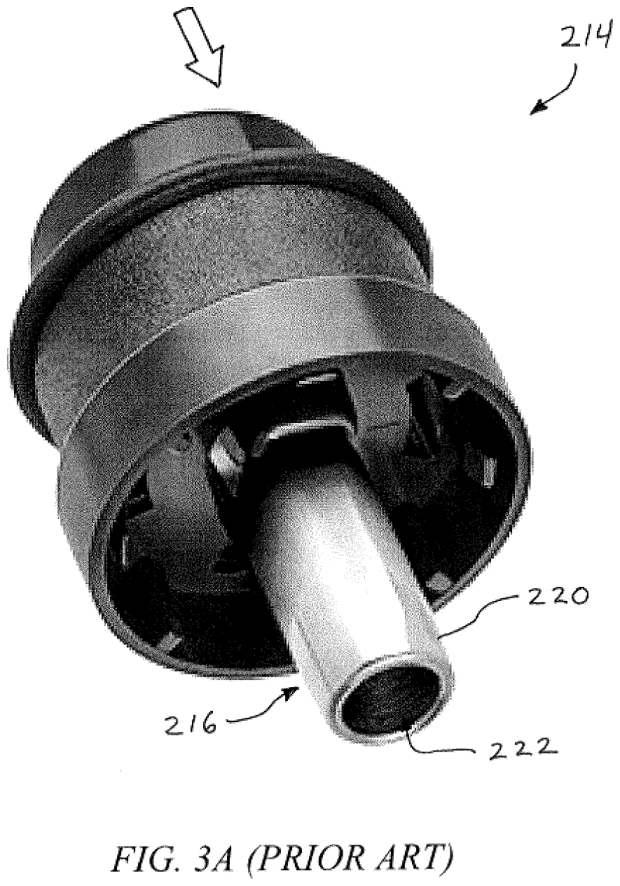

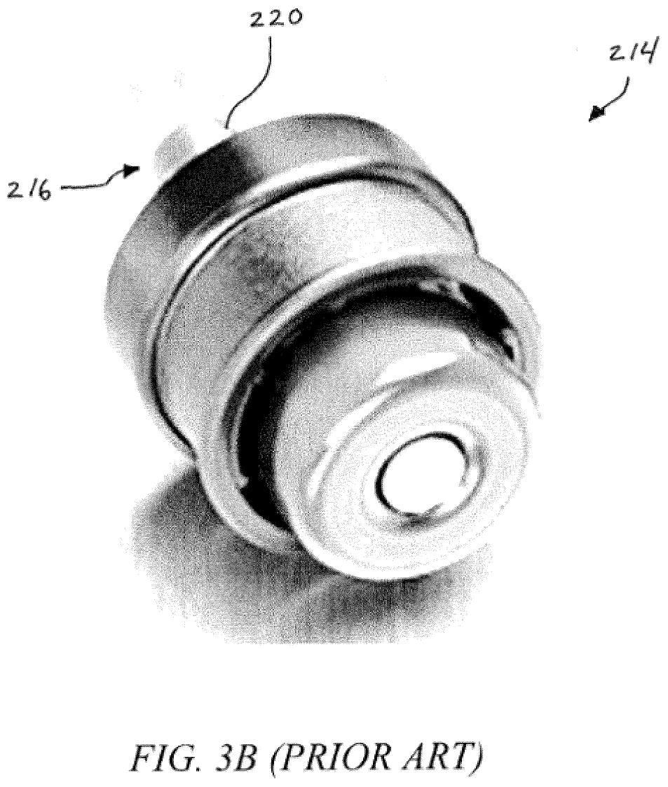

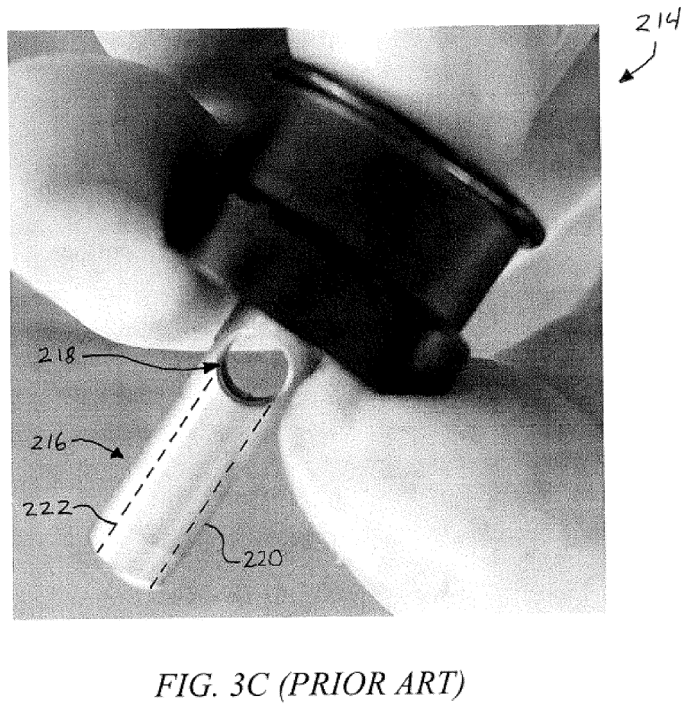

Referring now to FIGS. 2A-3C, conventional valves for controlling endoscopic suction are shown. A conventional endoscope 200 may include various valves 214 for controlling suction. Typical valves such as the valve 214 include a stem 216 having a shaft 220 (shown with reference to FIGS. 3A and 3C). In some conventional systems, the shaft 220 is accessible via an intermediate opening 218 positioned along the shaft 220, as well as a space between the stem 216 and a housing of the valve 214, which may be closed by way of the medical provider depressing the stem 216 as depicted with reference to FIG. 2B. The shaft 220 may be a hollow stem forming an internal duct 222 in communication with the opening 218. Thus, air may travel into the opening 218 and down through the internal duct 222. In other conventional systems, as shown with particular reference to a suction valve 214 depicted with reference to FIGS. 3A-3C, an intermediate opening 218 may be disposed along the valve stem 216. A suction device may be used to draw a flow from an interior passage 202 to a suction outlet 212. The valve 214 may control whether the flow is drawn from the suction inlet 208 or the space between the stem 216 and the housing of the valve 214 by moving the stem 216.

In one position shown in FIG. 2A, the valve 214 may be pressed down to position the stem 216 such that the intermediate opening 218 is aligned with a distal portion 204 of the interior passage 202 that interfaces with the suction inlet 208. As compared with the configuration depicted with reference to FIG. 2B, this changes the direction of the suction. The flow may be drawn from the suction inlet 208, through the distal portion 204, through the intermediate opening 218, through the internal duct 222 of the shaft 220, and to the suction outlet 212.

In another position shown in FIG. 2B, the valve 214 may be released to position the stem 216 such that the intermediate opening 218 is no longer aligned with the distal portion 204 of the interior passage 202. Rather, the distal portion 204 may now be blocked by a solid portion of shaft 220 of the stem 216. Further, air may travel through the space between the stem 216 and the housing of the valve 214, then through the internal duct 222 of the shaft 220 via the opening 218. In this case, due to the blockage of the distal portion 204 and the air travel through the internal duct 222, the suction flow may be drawn past the stem 216, and to the suction outlet 212, thereby applying no suction through the interior passage 202.

There are numerous issues involved with conventional systems that are embodied by the configurations depicted with reference to FIGS. 2A-2B. As a first example, the internal duct 222 of the shaft 220 may be subject to issues relating to clogging. As discussed above, the flow drawn from the suction inlet 208 must navigate the intermediate opening 218 and the internal duct 222 of the shaft 220. Especially in the case of the flow drawn from the suction inlet 208, the intermediate opening 218 and the internal duct 222 are subject to clogging, as the flow may contain bodily objects and other matter.

As a second example, the internal duct 222 of the shaft 220 and the intermediate opening 218 present efficiency issues relating to the endoscopic suction. In particular, the internal duct 222 of the shaft 220 (and, in some cases, the intermediate opening 218) must be formed with diameters that are less than a diameter of the interior passage 202. In this sense, a volumetric rate for the flow drawn through the internal duct of the shaft 220 and the opening 218 is necessarily less than that which could be drawn solely through the interior passage 202.

As a third example, control over the endoscopic suction may be limited in such conventional systems. As described above, airflow may be controlled by moving the intermediate opening 218 into alignment with the distal portion 204 of the interior passage 202. While the valve 214 may be operated as to only align a particular portion of the intermediate opening 218 with the distal portion 204, such control to reach particular rates of endoscopic suction may be difficult, and the binary nature of depressing the stem 216 in order to do so may limit the variable range of endoscopic suction that could otherwise be afforded.

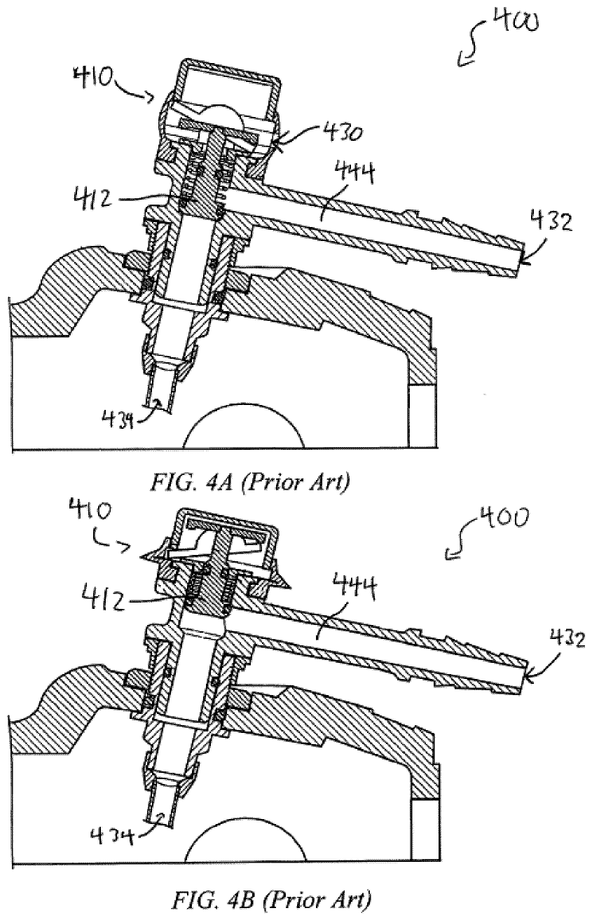

Referring now to FIGS. 4A-4B, another conventional valve for controlling endoscopic suction is shown. This conventional endoscope 400 may use a valve 410 for moving a plug 412 relative to an interior passage 444 that extends between a suction inlet 432 and a suction outlet 434 that interfaces with a suction device. When the plug 412 is in a first position shown in FIG. 4A that blocks the interior passage 444, a vent inlet 430 may be opened to allow air to travel from the vent inlet 430 to the suction outlet 434. When the plug 412 is moved to a second position shown in FIG. 4B, a flow is allowed to travel through the interior passage 444 and the vent inlet 430 is closed. While this conventional system may partially alleviate some of the issues relating to the internal duct 222 of the shaft 220 of the stem 216 (described above with reference to FIGS. 2A-3C), this conventional still presents issues relating to clogging and efficiency. For example, in order to close the vent inlet 430 and allow for endoscopic suction through the suction inlet 432, the plug 412 must be moved to the position shown in FIG. 4B, where the interior passage 444 is still at least partially obstructed, thereby reducing the optimal efficiency of endoscopic suction as opposed to a completely unobstructed interior passage. Moreover, even the partial obstruction of the interior passage 444 may result in significant clogging when the flow must navigate the contours of the plug 412 in order to travel to the suction outlet 434. Further, the plug 412 limits the effective diameter of the channel. In this sense, a volumetric rate for the flow drawn through the internal duct is necessarily less than that which could be drawn solely through the interior passage.

Referring now to FIGS. 5-9, embodiments of the valve 10 are shown, according to various embodiments of the present disclosure. As described above with reference to FIGS. 2A-4B, conventional systems present issues relating (at least) to clogging and efficiency. As described in greater detail below, the present disclosure provides an endoscope suction valve that can control endoscopic suction without obstructing an interior passage, while also leveraging a vent inlet in a manner that allows for advantageous dexterity for articulating a particular value of the flow for endoscopic suction.

Referring particularly to FIG. 5, the valve 10 of the apparatus 100 is shown in greater detail, according to some embodiments of the present disclosure. As shown, the valve 10 includes a housing 20 disposed on the endoscope 40, such that a plug 12 of the valve 10 is operable to engage the vent inlet 30. The valve 10 may include a stem 18 movably disposed within the housing 20. The stem 18 may be moved downwards (e.g., toward the vent inlet 30 and/or the interior passage 44) by pressing the button 16. When the button 16 is not being pressed to move the stem 18 downward, the stem 18 may be urged upwards (e.g., away from the vent inlet 30 and/or the interior passage 44) due to a biasing member 22 located between the button 16 and a top of the housing 20. Alternatively, the biasing member may be located at other suitable locations inside or on the body of the valve. The plug 12 may be disposed on a lower end of the stem 18, such that movement of the stem 18 articulates a position of the plug 12. In some embodiments, the plug 12 and the stem 18 are formed as a single component. In other embodiments, and as shown, the plug 12 and the stem 18 are separate components that are coupled together. In other embodiments still, the valve 10 is constructed such that the valve 10 does not include the stem 18, and the button 16 is directly attached to the plug 12. In some embodiments, the housing 20 includes an opening 14, such that a flow of room air may travel to the vent inlet 30.

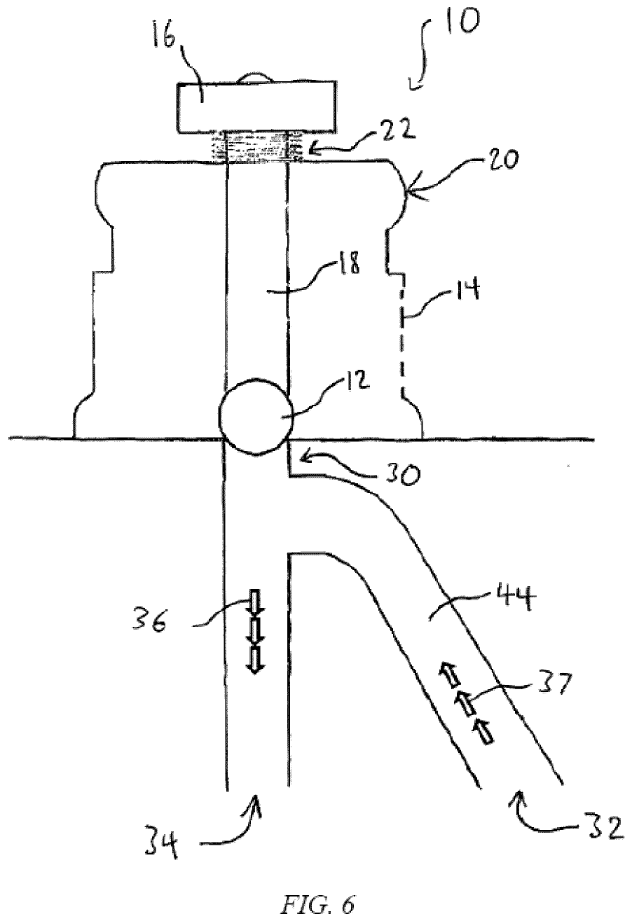

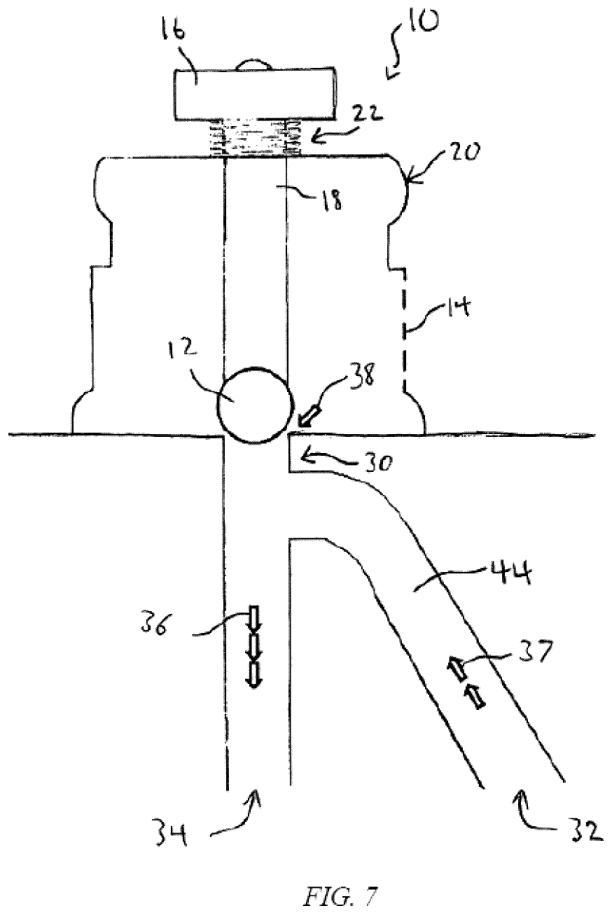

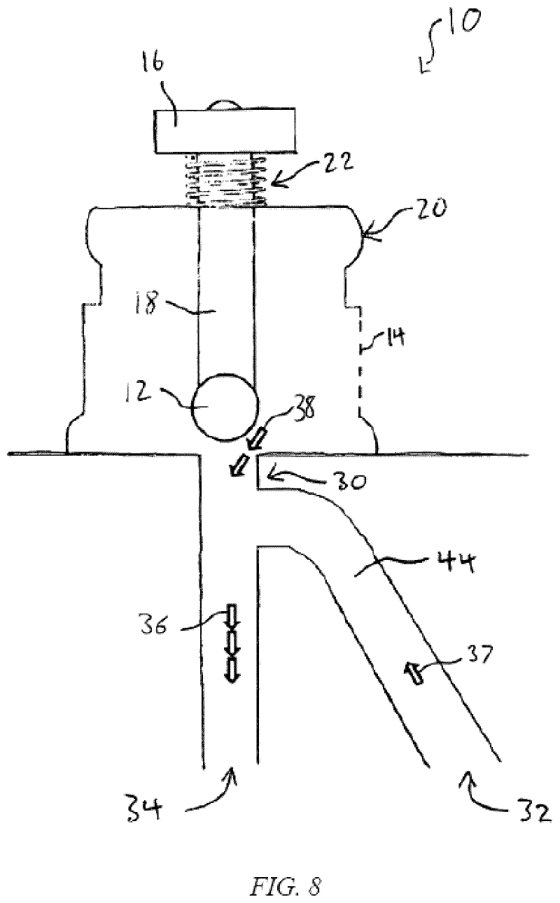

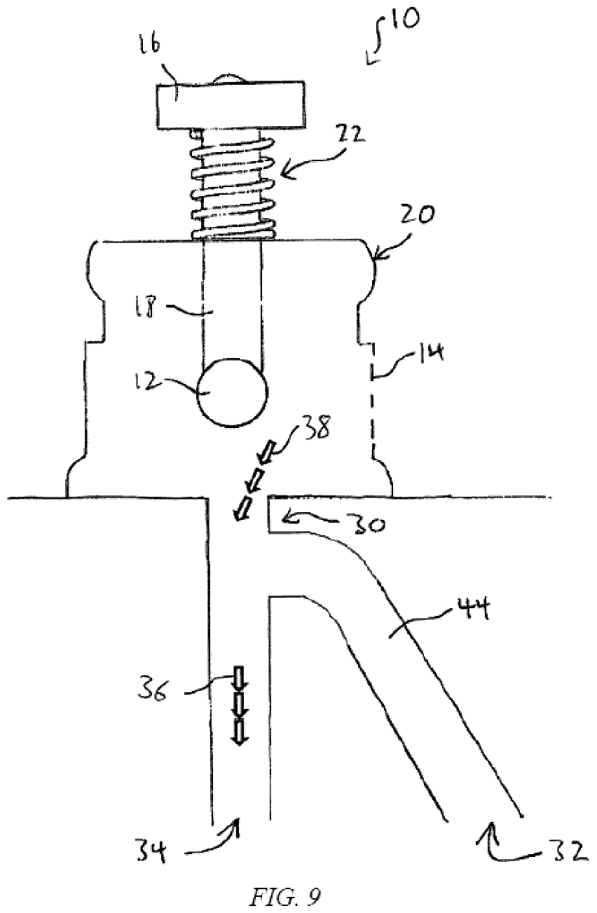

Referring again to FIGS. 5-9, the plug 12 of the valve 10 may be moved to various positions in order to control the amount of endoscopic suction being drawn from the suction inlet 32, according to various embodiments of the present disclosure. For example, FIGS. 5-6 show the valve 10 in a configuration where the plug 12 is in a first position, such that the plug 12 closes the vent inlet 30, flow is drawn through the suction inlet 32, through the interior passage 44, and to the suction outlet 34. In such configurations, no flow may be drawn through the vent inlet 30. As another example, FIG. 9 shows the valve 10 in a configuration where plug 12 is in a second position, such that the vent inlet 30 is open, flow is drawn through the vent inlet 30, through the interior passage 44, and to the suction outlet 34. In such configurations, no flow may be drawn through the suction inlet 32. As more examples still, FIGS. 7-8 show the valve 10 in configurations where the plug 12 is in third and fourth positions (respectively), such that the vent inlet 30 is opened, flow is drawn through the vent inlet 30, and flow is also being drawn from the suction inlet 32. Accordingly, the valve 10 may include the plug 12 which is movable between the first position and the second position. As shown, the plug 12 may be closer to the interior passage 44 when the plug 12 is in the first position than when the plug 12 is in the second position.

During the exemplary procedure mentioned above with reference to FIG. 1, the pump 52 may be constantly operated to draw a flow 36 from the interior passage 44 to the suction outlet 34. FIGS. 5-9 show, among other aspects of the present disclosure, that valve 10 is movable between the aforementioned first, second, third, and fourth positions to articulate a position of the plug 12 that controls from which inlet (the suction inlet 32 or the vent inlet 30) the flow 36 is drawn (see FIGS. 5, 6 and 9), or how much of the flow is drawn from each inlet when the flow 36 draws from each of them (see FIGS. 7-8). As depicted herein, the flow 36 drawn from the pump 52 may draw a first component 37 of flow from the suction inlet 32 and/or a second component 38 of flow from the vent inlet 30, depending on the configuration. It should be appreciated that in each configuration, the first component 37 and the second component 38, in combination, are substantially equivalent to the flow 36 being drawn by the pump 52. Thus, the following configurations are depicted to show the first component 37 and the second component 38 in order to illustrate the amount of endoscopic suction occurring at either inlet in order to satisfy the flow 36 being drawn to the suction outlet 34.

Accordingly, as discussed herein, a method of operating an endoscope (e.g., the apparatus 100) is provided. The method may include providing the body 40, which may form the passage wall the passage wall 42 that defines the interior passage 44 extending between the suction inlet 32 and the suction outlet 34. The method may further include providing the vent inlet 30 in fluid communication with the interior passage 44, and providing the valve 10 with the plug 12. The method may further include moving the plug 12 to the first position, such that the plug 12 closes the vent inlet 30 without occluding (e.g., blocking, clogging, obstructing, etc.) the interior passage 44. The method may further include moving the plug 12 away from the first position and to the second position, such that the vent inlet 30 is open.

Further, the disclosure herein thus provides for a method of retrofitting an endoscope (e.g., the apparatus 100). The method may include providing the valve 10 including the plug 12. The method may further include installing the valve 10 on an endoscope that includes the body 40, which may form the passage wall 42 that defines the interior passage 44 with a suction inlet 32 and the suction outlet 34. Of course, the endoscope may further include the vent inlet 30 in fluid communication with the interior passage 44. Thus, as retrofitted, the plug 12 may be movable to a first position where the plug 12 closes the vent inlet 30 without occluding the interior passage 44.

Referring particularly to FIGS. 5 and 6, the plug 12 is shown in the aforementioned first position where the plug 12 is seated over or within the vent inlet 30, such that the plug 12 closes the vent inlet 30, according to some embodiments of the present disclosure. As mentioned above, the pump 52 is configured to draw the flow 36 from the interior passage 44 to the suction outlet 34. When the plug 12 is positioned as shown, the entirety of the flow 36 is drawn from the suction inlet 32 and none of the flow 36 is drawn from the vent inlet 30, which is closed by the plug 12. Thus, the aforementioned first component 37 that is drawn from the suction inlet 32 makes up the entirety of, and is the same as, the flow 36 being drawn to the suction outlet 34.

In some embodiments, when the plug 12 is positioned as shown, the valve 10 is providing for a maximum suction configuration (in terms of the amount of flow being drawn from the suction outlet 32, and therefore the patient) for the endoscope apparatus 100. Such a configuration may provide for a standard amount of suction in endoscopic suction procedures, or may provide for an “extra” amount of suction in endoscopic suction procedures, while configurations such as the configuration depicted with reference to FIG. 7 represent a standard amount of suction.

When compared to conventional endoscopic valves such as those depicted with reference to FIGS. 2A-4B, it should be appreciated that the plug 12 is shown to not occlude the interior passage 44. In other words, when the plug 12 is in the first position, the plug 12 closes the vent inlet 30 without occluding the interior passage 44. Thus, a substantial amount of (or the entirety of) the interior passage 44 is available for the travel of the flow from the suction inlet 32 to the suction outlet 34, providing for endoscopic suction with an advantageous efficiency when compared to conventional systems. Moreover, by avoiding occlusion of the interior passage 44, clogging can be avoided.

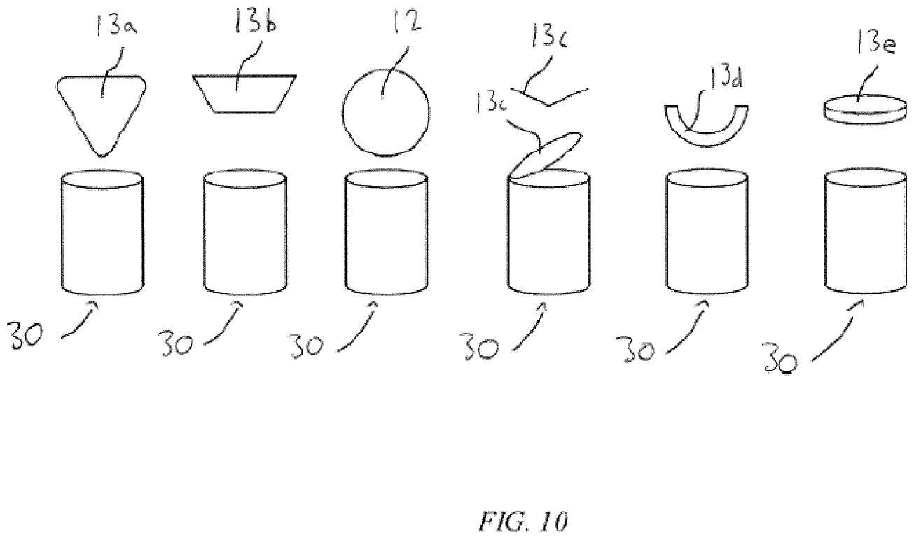

In some embodiments, and as shown with reference to FIG. 6 the vent inlet 30 defines a substantial passage length, such that the plug 12 engages the vent inlet 30 at a location that is substantially offset from the interior passage 44. Such a passage length may allow for closing of the valve inlet 30 without any obstruction of the interior passage 44 whatsoever, depending on the dimensions of the plug 12. In other embodiments, and as shown with reference to FIG. 5, the vent inlet 30 is simply an opening in the interior passage 44, such that the vent inlet does not define a substantial passage length and the plug 12 engages the vent inlet 30 at a location that is not substantially offset from the interior passage 44. Such a configuration may still allow for closing of the vent inlet 30 without occluding the interior passage 44 by adjusting the dimensions of the plug 12. Importantly, in the configurations depicted with reference to FIGS. 5 and 6, the plug 12 is configured to close the vent inlet without entirely occluding the interior passage 44. Moreover, while FIGS. 5 and 6 show the plug 12 is generally depicted herein as spherical, the plug 12 can be constructed in a number of shapes, such as a disk or a flap, as shown with reference to FIG. 10.

In some embodiments, the edges of the vent inlet 30 form right-angles (or approximately so) as shown. In other embodiments, the edges of the vent inlet 30 are chamfered. In other embodiments still, the edges of the vent inlet 30 are curved. It should be appreciated that the edges vent inlet 30 may be formed in any manner that allows the plug 12 to form a close with the vent inlet 30.

Referring particularly to FIG. 7, the plug 12 is shown in the aforementioned third position where the plug 12 has been moved upwards (compared to the position of the plug 12 shown with reference to FIG. 6), such that the plug 12 is no longer seated over or within the vent inlet 30, according to some embodiments of the present disclosure. For example, the plug 12 may be movable between the first and third positions. As shown, the vent inlet 30 is no longer closed, and the flow 36 being drawn to the suction outlet 34 is no longer being provided entirely by the suction inlet 32 (as depicted by the first component 37). In some embodiments, this scenario is a result of a distance between the vent inlet 30 and the suction outlet 34 being less than a distance between the vent inlet 30 and the suction inlet 32. Due to this difference in distance, air may be drawn easier (e.g., with less of a pressure drop) from the vent inlet 30 than the suction inlet 32. Accordingly, when the path that the first portion 38 travels along through the housing 20 is unobstructed to some degree, the flow 36 being drawn to the suction outlet 34 begins to draw, at least partially, from room air via the vent inlet 30 (as depicted by the second component 38). Due to the flow 36 being satisfied, at least partially, by the second component 38, the first component 37 decreases accordingly, resulting in a decrease in endoscopic suction being drawn from the suction inlet 32. Thus, moving the plug 12 from the second position to the third position may increase the first component 37 of the flow 36 and decrease the second component 38 of the flow 36; similarly, moving the plug 12 from the first position to the third position may decrease the first component 37 of the flow 36 and increase the second component 38 of the flow 36.

When compared to conventional endoscopic valves such as those depicted with reference to FIGS. 2A-4B, it should be appreciated that the plug 12 only needs to be slightly adjusted to open the vent inlet 30 and thus decrease the amount of endoscopic suction being drawn from the suction inlet 32. This provides for an advantageous amount of dexterity over articulating an amount of endoscopic suction when compared to such conventional systems that must abruptly open the vent inlet 30 (or with substantial inefficiency, at least) in order to decrease or stop endoscopic suction.

Referring particularly to FIG. 8, the plug 12 is shown in the aforementioned fourth position where the plug 12 has been moved further upwards (compared to the position of the plug 12 shown with reference to FIG. 7), such that the plug 12 is further elevated away from the vent inlet 30, according to some embodiments of the present disclosure. For example, the plug 12 may be movable between the first, third, and fourth positions. By moving the plug 12 further upwards, the second component 38 may increase relative to the configuration depicted with reference to FIG. 7. Such an increase may be a result of the plug 12 providing less obstruction between the vent inlet 30 and the opening 14 as the plug 12 is moved further upwards and further away form a path that the second component 38 travels along through the housing 20 of the valve 10.

Given the constant volumetric rate of flow drawing the flow 36 to the suction outlet 34, the first component 37 simultaneously decreases relative to the configuration depicted with reference to FIG. 7. Thus, in some embodiments, the decrease to the first component 37 and the increase to the second component 38 bear a proportional relationship. Of course, moving the plug 12 downwards and toward the vent inlet 30 (moving from the configuration depicted with reference to FIG. 8 to the configuration depicted with reference to FIG. 7, as an example) may reverse the proportional relationship, such that the first component 37 increases while the second component 38 decrease. Juxtaposing the configurations depicted with reference to both FIGS. 6 and 7, the endoscope apparatus 100 provided for herein when operated using the valve 10 is further shown to allow for advantageously dexterous control over a variable amount of endoscopic suction being drawn through the suction inlet 32, simply depending on the position of the plug 12 as articulated by the movement of the stem 18 and/or the button 16.

Referring particularly to FIG. 9, the plug 12 is shown in the aforementioned second position where the plug 12 has been moved further upwards (compared to the position of the plug 12 shown with reference to FIG. 8), such that the plug 12 is further elevated away from the vent inlet 30, according to some embodiments of the present disclosure. For example, the plug 12 may be movable between the first, second, third, and fourth positions. By moving the plug 12 further upwards and completely un-obstructing (or un-obstructing to a threshold degree) the path of the second portion 38 through the housing 20, the second portion 38 may increase to a maximum amount (e.g., a flow rate that is equivalent to the entirety of the flow 36 being drawn to the suction outlet 34 by the pump 52), such that the first portion 37 is eliminated and no endoscopic suction is drawn from the suction inlet 32 (or substantially so).

In some embodiments, this scenario is a result of the aforementioned difference between the distance between the vent inlet 30 and the suction outlet 34 as opposed to the distance between the vent inlet 30 and the suction inlet 32. Due to the pressure drop associated with air being drawn via the second component 38 being less than the air being drawn via the first component 37 (as discussed above), the plug 12 may be articulated as shown to reach a point where the entirety of the flow 36 is drawn via the second component 38, thus eliminating the first component 37.

When compared to conventional endoscopic valves such as those depicted with reference to FIGS. 2A-4B, it should be appreciated that, similar to the second component 38 being allowed to travel through the unobstructed interior passage 44 when being drawn from the suction inlet 32, the first component is similarly able to be drawn through the unobstructed interior passage 44, further providing advantageous efficiency of the apparatus 100 in terms of allowing the first component 37 to be drawn to a degree that ceases endoscopic suction via the second component 38 form the suction inlet 32.

Referring to FIG. 10, various alternative embodiments of the plug 12 are shown. As mentioned above, while the plug 12 is generally depicted herein as spherical, the plug 12 can be constructed in a number of alternative shapes. As a first example, the plug 12 may be replaced by a plug 13a which forms a cone. In some embodiments, the plug 13a may be positioned such that an apex of the cone faces the valve inlet 30 and/or the interior passage 44. As a second example, the plug 12 may be replaced by a plug 13b which forms a frustrated cone. In some embodiments, the plug 13b may be positioned such that a narrow end of the frustrated cone faces the valve inlet 30 and/or the interior passage 44.

As a third example, the plug 12 may be replaced by a plug 13c which forms a flap. For example, the flap may be disposed on or about the body 40 via a hinge which allows the flap to close toward or open away from the vent inlet 30. In this case, the aforementioned passage length defined by the valve inlet 30 may not be necessary, as no portion of the valve inlet 30 would need to be occluded in order to close the valve inlet 30 (as opposed to the use of the plug 12 or the plug 13A, for example). As a fourth example, the plug 12 may be replaced by a plug 13d which forms a diaphragm. As a fifth example, the plug 12 may be replaced by a plug 13e which forms a disk which, similar to the plug 13c, may obviate the need for a passage length of the valve inlet 30.

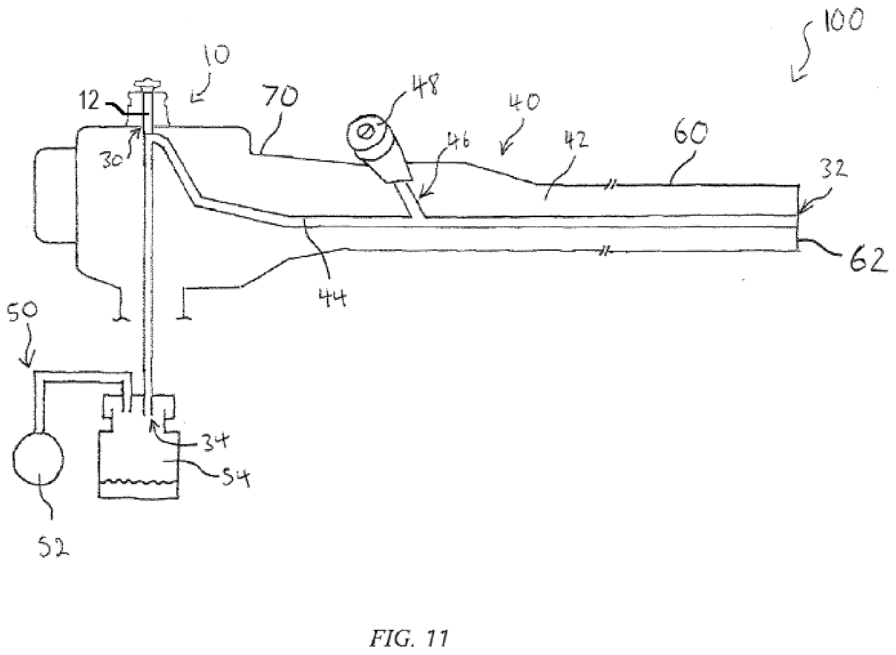

Referring now to FIG. 11, the apparatus 100 is shown, according to further embodiments of the present disclosure. As shown, the plug 12 may be a small stem (similar to the stem 18 as described herein) that is long enough such that when the plug 12 is seated over or within the vent inlet 30 (as described with reference to FIGS. 5 and 6) in order to close the vent inlet 30, a portion of the plug 12 is positioned within the vent inlet 30 without blocking the interior passage 44 extending between the suction inlet 32 and the suction outlet 34. Advantageously, such embodiments may provide a more secure seal of the vent inlet 30.

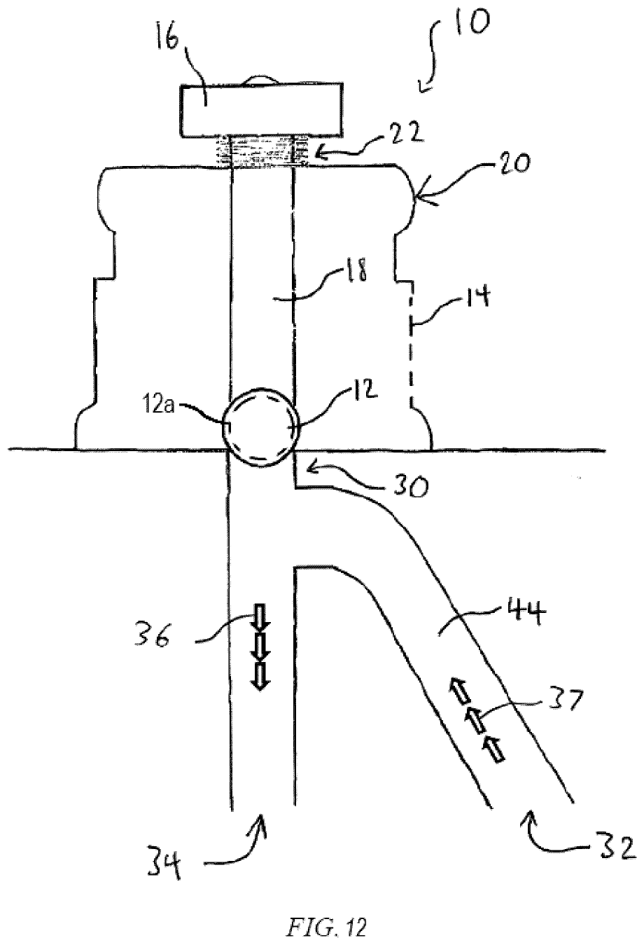

Referring now to FIG. 12, the apparatus 100 is shown, according to further embodiments of the present disclosure. In some embodiments, the plug 12 includes a coating 12a. For instance, the coating 12a may be a silicone or foam coating that surrounds the plug 12.

As mentioned above with reference to FIGS. 5-9, the plug 12 of the valve 10 may be moved to various positions in order to control the amount of endoscopic suction being drawn from the suction inlet 32. FIG. 12 generally depicts the apparatus 100 in a configuration corresponding to FIG. 6 (e.g., when the plug 12 is seated over or within the vent inlet 30 in order to close the vent inlet 30).

In some cases, if both the plug 12 and the vent inlet 30 are made of hard materials, the seal formed between the plug 12 and the vent inlet 30 may be difficult to maintain (e.g., due to a lack of deformation of the surface of the plug 12 against the vent inlet 30 or vice-versa), which may prevent optimal suction. However, the coating 12a may allow for such deformation, thus providing an advantageous seal between the plug 12 and the vent inlet 30. Moreover, the soft material of the coating 12a may allow for enhanced adjustment of the seal between the plug 12 and the vent inlet 30 based on how hard the plug 12 is pressed into the vent inlet 30 (which may be afforded due to the slight deformation of the coating 12a against the vent inlet 30). Accordingly, the coating 12a may provide for more fine control of the suction provided by the apparatus 100. In further embodiments, changing the thickness or hardness of the coating 12a may change how much force is required to get a full seal between the plug 12 and the vent inlet 30 (e.g., a thicker and/or softer coating 12a may allow for less force required to obtain a full seal between the plug 12 and the vent inlet 30).

Thus, although there have been described particular embodiments of the present invention of a new and useful ENDOSCOPE SUCTION VALVE, it is not intended that such references be construed as limitations upon the scope of this invention.

What is claimed is:

1. An endoscope apparatus, comprising:
a body forming a passage wall that defines an interior passage extending between a suction inlet and a suction outlet;
a vent inlet in fluid communication with the interior passage; and
a valve having a plug that is movable between a first position and a second position,
wherein when the plug is in the first position, the plug closes the vent inlet without occluding the interior passage, and
wherein when the plug is in the second position, the vent inlet is open.

2. The apparatus of claim 1, wherein the plug is closer to the interior passage when the plug is in the first position than when the plug is in the second position.

3. The apparatus of claim 1, further comprising a pump in fluid communication with the suction outlet, wherein the pump draws a flow from the interior passage to the suction outlet.

4. The apparatus of claim 3, wherein when the plug is in the first position, the flow travels from the suction inlet, through the interior passage, and to the suction outlet.

5. The apparatus of claim 3, wherein when the plug is in the second position, the flow travels from the vent inlet, through the interior passage, and to the suction outlet.

6. The apparatus of claim 5, wherein when the plug is in the second position, the flow does not travel from the suction inlet, through the interior passage, and to the suction outlet.

7. The apparatus of claim 3, wherein the plug is further movable to a third position between the first and second positions, and
wherein when the plug is in the third position, a first component of the flow travels from the suction inlet to the suction outlet and a second component of the flow travels from the vent inlet to the suction outlet.

8. The apparatus of claim 7, wherein moving the plug from the second position to the third position increases the first component of the flow and decreases the second component of the flow.

9. The apparatus of claim 7, wherein moving the plug from the first position to the third position decreases the first component of the flow and increases the second component of the flow.

10. The apparatus of claim 1, wherein the plug forms a cone, such that an apex of the cone faces the interior passage.

11. The apparatus of claim 11, wherein the cone is a frustrated cone, such that an apex of the cone faces the interior passage.

12. The apparatus of claim 1, wherein the plug forms a sphere.

13. The apparatus of claim 1, wherein the plug forms a diaphragm.

14. The apparatus of claim 1, wherein the plug forms a disk.

15. The apparatus of claim 1, wherein the plug forms a flap.

16. A method of operating an endoscope, comprising:
providing a body forming a passage wall that defines an interior passage extending between a suction inlet and a suction outlet;
providing a vent inlet in fluid communication with the interior passage;
providing a valve with a plug;
moving the plug to a first position, such that the plug closes the vent inlet without occluding the interior passage; and
moving the plug away from the first position and to a second position, such that the vent inlet is open.

17. The method of claim 16, further comprising proving a pump in fluid communication with the suction outlet,
wherein the pump draws a flow from the interior passage to the suction outlet,
wherein when the plug is in the first position, the flow travels from the suction inlet, through the interior passage, and to the suction outlet, and
wherein when the plug is in the second position, the flow travels from the vent inlet, through the interior passage, and to the suction outlet.

18. The method of claim 17, wherein while the plug is being moved away from the first position and to the second position and is between the first position and the second position, a first component of the flow travels from the suction inlet to the suction outlet and a second component of the flow travels from the vent inlet to the suction outlet, and
wherein while the plug is being moved away from the first position and to the second position and is between the first position and the second position, the first component of the flow decreases and the second component of the flow increases.

19. The method of claim 18, wherein when the plug is in the second position, the flow does not travel through the suction inlet, through the interior passage, and to the suction outlet.

20. A method of retrofitting an endoscope, comprising:
providing a valve including a plug; and
installing the valve on an endoscope that includes a body forming a passage wall that defines an interior passage with a suction inlet and a suction outlet,
wherein the endoscope further includes a vent inlet in fluid communication with the interior passage,
wherein the plug is movable to a first position where the plug closes the vent inlet without occluding the interior passage.
